(19) United States
(12) Patent Application Publication
(10) Pub. No.: US 2026/0166267 A1
(43) Pub. Date: Jun. 18, 2026

(54) BORON NITRIDE-TREATED POLYIMIDE COATING

(71) Applicant: ZEUS COMPANY LLC, Orangeburg, SC (US)

(72) Inventors: Charles Wayne Conley, Dunlap, TN (US); Ian W. Metcalfe, Hixon, TN (US); Ravish K. Akhani, Lexington, SC (US)

(21) Appl. No.: 19/418,747

(22) Filed: Dec. 12, 2025

Related U.S. Application Data
(60) Provisional application No. 63/733,727, filed on Dec. 13, 2024.

(51) Int. Cl. A61M 25/00; C09D 133/24

(52) U.S. Cl. CPC A61M 25/0045; C09D 133/24

(57) ABSTRACT

Increasingly, nonfluorinated polymers are needed to replace fluorinated materials in catheter liners and catheter constructions. Therefore, it would be advantageous for non-fluorinated materials to achieve sufficient lubricity and mechanical strength for such applications (e.g., comparable to fluorinated materials). A boron nitride-treated polyimide freestanding tube that can be employed as a coating is described herein for such purposes.

### CROSS-REFERENCE TO RELATED APPLICATIONS

The present application claims priority to U.S. Provisional Patent Application No. 63,733,727, filed Dec. 13, 2024, the disclosure of which is incorporated herein by reference in its entirety.

### FIELD OF THE INVENTION

The present disclosure relates to coatings suitable for association with tubings such as catheter liners. Coatings (e.g., in freestanding form), coated tubes, as well as methods for preparing the coatings, coated tubes, and constructions/assemblies (e.g., catheters) comprising such tubes are provided herein as well.

### BACKGROUND

Polytetrafluoroethylene (PTFE) has been the ideal material for inner liners of catheters due to the chemical resistance, biocompatibility, and low coefficient of friction (COF) of PTFE. PTFE exhibits unique characteristics in this field that other polymers do not exceed. With such a low COF, PTFE has been able to provide an inner diameter (ID) suitable for catheter liners that easily allows various catheter technologies such as stents, balloons, and atherectomy or thrombectomy devices to be pushed through a small diameter catheter lumen. The effect of low COF/increased lubricity of the catheter inner diameter is a reduced deployment force of catheter devices as the catheter devices are passed through the lumen of the catheter ID (inner diameter), increasing the likelihood of a successful procedure.

In catheter constructions, the catheter liner is stretched over a mandrel, which is usually stainless steel or PTFE. A braided or coiled wire reinforcing layer may be constructed on top of the liner and can vary in picks, wire dimensions, and materials for different applications. Stainless steel or nitinol hypotubes are also used in neurovascular applications. A catheter jacket is then slid over the underlying layers, followed by a heat shrink tube over the catheter jacket. A thermoplastic tie layer made typically of the same material and durometer as the catheter jacket can be deposited on the surface of the catheter liner for enhanced adhesion. The finalized construction is then laminated together and removed from the mandrel, resulting in a fully built catheter.

Increasingly, there is a need to find alternatives to using fluorinated polymer materials in catheter constructions due to environmental concerns related to per- and polyfluoroalkyl substances (PFAS). Ideally, an alternative material achieves comparable lubricity and mechanical properties as the fluorinated materials that are used most commonly in current catheter constructions. The need for comparable lubricity is particularly important for catheter liners.

Polyimide (PI) tubing is used within the catheter market primarily as a liner due to its high-performance in terms of chemical, mechanical, and thermal characteristics. The combination of these properties along with the fabrication of thin-walled, high-strength structures, makes polyimide a material of choice for advanced medical device applications. However, a particular limitation of conventional polyimide is its relatively high coefficient of friction compared to polytetrafluoroethylene (PTFE), a parameter that is critical to catheter performance and patient comfort, particularly in applications requiring low insertion force and enhanced lubricity.

To address this limitation, PTFE fillers have traditionally been incorporated into polyimide formulations to reduce surface friction and improve trackability. One example of such a product offering is PI Glide™ from Zeus Company LLC. Although PI liners that incorporate PTFE fillers can exhibit highly desirable attributes such as exceptional lubricity, chemical inertness, and biocompatibility, one performance disadvantage of current PI-PTFE materials is that the mechanical properties of the PI are generally diminished at a certain point as the concentration of PTFE fillers increases, meaning in general as lubricity increases, mechanical strength decreases.

Furthermore, the use of these materials is increasingly constrained by global regulatory pressure aimed at reducing or eliminating per- and polyfluoroalkyl substances (PFAS). These regulatory changes stem from heightened awareness of the environmental persistence, potential toxicity, and long-term health implications associated with fluorinated compounds. Therefore, it is necessary to develop a robust non-fluorinated material alternative, and it would be ideal if such a material could achieve enhanced lubricity without compromising the mechanical strength of polyimides. Such a material would maximize catheter performance and patient comfort and safety while providing a more environmentally sustainable, PFAS-free, solution that meets emerging regulatory and market needs.

### SUMMARY OF THE INVENTION

The disclosure provides coating materials, coated products (e.g., tubings) comprising such coating materials, and assemblies incorporating such coated products, e.g., catheters. The coating materials, as described herein, generally comprise boron nitride-treated polyimide. Advantageously, such coating materials can provide alternatives to fluorinated materials commonly used in catheter liners and catheter assemblies/constructions.

The disclosure provides, without limitation, the following embodiments:

Embodiment 1: A tube comprising boron nitride-treated polyimide, the tube having an inner surface and an outer surface, wherein tube exhibits: a) a coefficient of friction (COF) less than 1.6 on the inner surface and the outer surface; b) a Ra surface roughness less than 12 microinches on the inner surface and the outer surface; c) a Rz surface roughness less than 70 microinches on the inner surface and the outer surface; and d) a tensile modulus between 400-600 Ksi.

Embodiment 2: The tube of Embodiment 1, wherein the tube is substantially homogeneous in composition.

Embodiment 3: The tube of Embodiment 1 or 2, comprising about 5% to about 25% boron nitride by weight, based on a weight of the tube.

Embodiment 4: The tube of Embodiment 1 or 2, comprising about 10% to about 20% boron nitride by weight (e.g., about 15% by weight), based on a weight of the tube.

Embodiment 5: The tube of any of Embodiments 1-4, having an average wall thickness of 0.0005 to 0.002″.

Embodiment 6: The tube of any of Embodiments 1-5, consisting essentially of the boron nitride-treated polyimide.

Embodiment 7: The tube of any of Embodiments 1-5, further comprising one or more fillers.

Embodiment 8: The tube of any of Embodiment 1-7, wherein the COF is less than 1.3.

Embodiment 9: The tube of any of Embodiments 1-7, wherein the COF is less than 1.2.

Embodiment 10 A catheter comprising the tube of any of Embodiments 1 on an inner diameter thereof.

Embodiment 11: A coated liner, comprising: a liner tubing; and a coating comprising the tube of any of Embodiments 1-9 on at least a portion of an inner surface of the liner tubing and/or the outer surface of the liner tubing.

Embodiment 12: The coated liner of Embodiment 11, wherein the tube is in a radially unstretched form.

Embodiment 13: The coated liner of Embodiment 11, wherein the tube is in a radially stretched form.

Embodiment 14: The coated liner of any of Embodiments 11-13, wherein the liner tubing comprises a non-fluorinated polymeric material.

Embodiment 15: The coated liner of any of Embodiments 11-14, wherein the coating is present only on at least a portion of the inner surface of the liner tubing.

Embodiment 16: The coated liner of any of Embodiments 11-14, wherein the coating is present only on at least a portion of the outer surface of the liner tubing.

Embodiment 17: The coated liner of any of Embodiments 11-14, wherein the coating is present on at least a portion of the outer surface of the liner tubing and at least a portion of the inner surface of the liner tubing (wherein the coating on at least a portion of the outer surface of the liner tubing comprises one tube of any of Embodiments 1-9 and wherein the coating on at least a portion of the inner surface of the liner comprises another tube of any of Embodiments 1-9, and wherein such tubes can be the same, e.g., in composition and/or thickness or can be different).

Embodiment 18: The coated liner of any of Embodiments 11-17, wherein the coating is present on substantially all of the inner surface of the liner tubing and/or the outer surface of the liner tubing.

Embodiment 19: The coated liner of any of Embodiments 11-18, further comprising one or more different coatings in addition to the coating comprising boron nitride-treated polyimide.

Embodiment 20: A catheter comprising the coated liner of any of Embodiments 11-18 on an inner diameter thereof.

These and other features, aspects, and advantages of the disclosure will be apparent from a reading of the following detailed description, together with the accompanying drawings, which are briefly described below. The invention includes any combination of two, three, four, or more of the above-noted embodiments, as well as combinations of any two, three, four, or more features or elements set forth in this disclosure, regardless of whether such features or elements are expressly combined in a specific embodiment description herein. This disclosure is intended to be read holistically such that any separate features or elements of the disclosed invention, in any of its various aspects and embodiments, should be viewed as intended to be combinable unless the context clearly dictates otherwise.

### DETAILED DESCRIPTION

The present invention now will be described more fully hereinafter with reference to the accompanying drawings, in which some, but not all embodiments of the inventions are shown.

Indeed, these inventions may be embodied in many different forms and should not be construed as limited to the embodiments set forth herein; rather, these embodiments are provided so that this disclosure will satisfy applicable legal requirements. Like numbers refer to like elements. As used in this specification and the claims, the singular forms “a,” “an,” and “the” include plural referents unless the context clearly dictates otherwise.

A non-fluoropolymer coating material is provided herein, which comprises a boron nitride-treated polyimide. In some embodiments, the boron nitride-treated polyimide comprises boron nitride (BN) that has been mixed with and/or reacted with a polyimide precursor (e.g., polyamic acid (PA)). In other words, in some embodiments, the coating material can be described as comprising, consisting essentially of, or consisting of a mixture of and/or a reaction product of BN and PA, and/or can be described as comprising, consisting essentially of, or consisting of a boron nitride-treated polyimide. Advantageously, in certain embodiments, the coating material is substantially free of any fluoropolymer, meaning that no fluoropolymer is incorporated within the coating material.

BN is generally provided in the form of particles, and the BN used in the production of the coating materials described herein can have various sizes and shapes. Certain non-limiting shapes of BN are platelets (typically having smaller particle sizes) and agglomerates (typically having larger particle sizes). Particle sizes of BN range, in some embodiments, from about 0.5 d50 μm to about 200 d50 μm. Certain non-limiting ranges of suitable particle sizes are, for example, 0.5 d50 μm to 200 d50 μm, 0.8 d50 μm to 200 d50 μm, 0.8 d50 μm to 100 d50 μm, 0.8 d50 μm to 80 d50 μm, 0.8 d50 μm to 50 d50 μm, 0.8 d50 μm to 25 d50 μm, 0.8 d50 μm to 20 d50 μm, 0.8 d50 μm to 10 d50 μm, 0.8 d50 μm to 8 d50 μm, 0.8 d50 μm to 6 d50 μm, 1 d50 μm to 200 d50 μm, 1 d50 μm to 100 d50 μm, 1 d50 μm to 80 d50 μm, 1 d50 μm to 50 d50 μm, 1 d50 μm to 25 d50 μm, 1 d50 μm to 20 d50 μm, 1 d50 μm to 10 d50 μm, 1 d50 μm to 8 d50 μm, 1 d50 μm to 6 d50 μm, 25 d50 μm to 200 d50 μm, 25 d50 μm to 100 d50 μm, 25 d50 μm to 80 d50 μm, 50 d50 μm to 200 d50 μm, 50 d50 μm to 100 d50 μm, 50 d50 μm to 80 d50 μm, 100 d50 μm to 200 d50 μm, or 100 d50 μm to 150 d50 μm. In some specific embodiments, the BN is in platelet form, with a particle size of 50, 40, or 30 d50 μm or less. In some specific embodiments, the BN is in agglomerate form, with a particle size of 50, 60, 70, or 75 d50 μm or greater.

The content of BN in the coating material can vary and, in some embodiments, may be 5-50% by weight, e.g., based on the weight of the boron nitride-treated polyimide. For example, in some embodiments, the content of BN can be 1% to 50%, 2% to 50%, 3% to 50%, 4% to 50%, 1% to 25%, 1% to 10%, 2% to 25%, 2% to 10%, 3% to 25%, 3% to 10%, 4% to 25%, 4% to 10%, 5% to 50%, 10% to 50%, 15% to 50%, 20% to 50%, 25% to 50%, 30% to 50%, 40% to 50%, 5% to 40%, 5% to 30%, 5% to 25%, 5% to 20%, 5% to 15%, 5% to 10%, 10% to 40%, 10% to 30%, 10% to 25%, 10% to 20%, 20% to 40%, 20% to 30%, 20% to 25%, 25% to 40%, 25% to 30%, 30% to 50%, or 30% to 40% by weight, based on the weight of the boron nitride-treated polyimide.

As noted, the boron nitride-treated polyimide coating material can, in some embodiments consist or consist essentially of the referenced boron nitride-treated polyimide. As such, in some embodiments, the remainder of the coating material in some embodiments comprises PI (e.g., about 50-95% by weight, e.g., based on the weight of the boron nitride-treated polymide.

In some embodiments, the boron nitride-treated polyimide coating material can comprise one or more further components. Where further components are present in the boron nitride-treated polyimide coating material, such components can include, for example, unreacted PA, unreacted BN, solvent, and the like. In preferred embodiments, the boron nitride-treated polyimide coating material is substantially free of solvent, unreacted BN, and/or unreacted PA.

In some embodiments, further components optionally present within the boron nitride-treated polyimide coating material can include other polymers and/or fillers. In some embodiments, no fillers are included within the disclosed boron nitride-treated polyimide coating material. In some embodiments, less than 1% by weight or less than 1% by volume of fillers are included in the disclosed boron nitride-treated polyimide coating material. Typical fillers can include, e.g., fibrous and non-fibrous materials. In some embodiments, the disclosed boron nitride-treated polyimide coating material can comprise higher amounts of fillers (e.g., up to 50% by weight of up to 50% by volume); in some embodiments, such fillers (e.g., when included in these higher amounts) are “non-fibrous” fillers. Advantageously, the coating material (e.g., provided in the form of a freestanding tube, as described herein below) and in some embodiments, the coated product (e.g., coated tubing) and, in some embodiments, an entire coated catheter liner and/or construction comprising such a coated product) contains substantially no fluoride-containing components.

In some embodiments, the boron nitride-treated polyimide coating material is in the form of a freestanding tube. The freestanding coating tubes can, in some embodiments, be substantially homogeneous, comprising, consisting essentially of, or consisting of the boron nitride-treated polyimide as previously described (optionally in combination with one or more fillers, polymers, etc. as described elsewhere herein). The freestanding coating is advantageously is lubricous on its inner diameter (ID) and its outer diameter (OD).

The freestanding tube comprising the boron nitride-treated polyimide coating material can be described, e.g., by its surface roughness, e.g., Ra and/or Rz (which values are typically substantially the same on the outer and inner surfaces thereof). In manufacturing, surface roughness refers to the deviation in the texture of a material's surface from its ideal surface. Surface roughness can be quantified with several parameters, including Ra (Roughness Average) and Rz (Ten-point Height).

Ra, also known as “average roughness,” is the average height of the texture of a material's surface. Essentially, Ra tells you how bumpy or smooth a surface is by calculating the average height of the peaks and valleys of a surface's texture. Ra allows manufacturers to quickly discern whether a material or part is rough or smooth enough for a given application. In some embodiments, the Ra of the inner and/or outer surface of the freestanding tube is less than 20 microinches, less than 18 microinches, less than 15 microinches, or less than 12 microinches, e.g., less than 11, less than 10, less than 9, less than 8, less than 7, less than 6, less than 5, or less than 4 microinches, e.g., about 1 to about 12 microinches.

Rz, also known as “Ten-point Height of Irregularities,” is the average difference between the five highest peaks and the five deepest valleys of a surface's texture. Compared to Ra, Rz provides a better view of the outlier peaks and valleys on a surface. This is especially beneficial for applications in which these outliers could affect functionality, such as seals or sliding parts or lubricious materials. In some embodiments, the Rz of the inner and/or outer surface of the freestanding tube is less than 100 microinches, less than 70 microinches, less than 60 microinches, less than 50 microinches, less than 40 microinches or less than 30 microinches, e.g., about 20 to about 100 microinches or about 20 to about 60 microinches.

While Ra provides an idea of the overall average roughness of a material's surface, Rz delves into specific irregularities. Calculating both and using them together is beneficial and helps give a more comprehensive understanding of a material's surface. Methods of obtaining such values are described herein below in the experimental section.

In some embodiments, the freestanding tube can be characterized by its coefficient of friction (which is typically substantially the same on the outer and inner surfaces thereof). COF and methods of obtaining COF are known in the art and mentioned in the examples herein below. In some embodiments, the COF of the inner and/or outer surface of the freestanding tube is less than 1.6; in some embodiments, the COF is even lower, e.g., less than 1.5, less than 1.4, less than 1.3, less than 1.2, less than 1.1, or less than 1.0.

In some embodiments, the freestanding tube can be characterized by its tensile properties, e.g., modulus, elongation, and/or tensile strength. Such parameters and methods of obtaining such values are known in the art and mentioned in the examples herein below. In some embodiments, the tensile modulus of the freestanding tube is 400-600 KSI, e.g., 400 to 500 KSI or 500 to 600 KSI; in some embodiments, the elongation is about 20 to about 70%; and in some embodiments, the tensile strength is 12,000 to 25,000 psi, e.g., 15,000 to 20,000 psi.

Advantageously, the boron nitride-treated polyimide coating material described herein can be in the form of a layer on at least a portion of a surface of a product, e.g., a tubing (e.g., a “liner tubing”). Although not intending to be limiting, the freestanding tube described herein above can be applied to an inner diameter and/or an outer surface of the product, e.g., tubing to produce such a coating/layer thereon. Such application can involve stretching (e.g., radially stretching) the freestanding tube over a surface, thereby forming a coating on that surface, or can be applied without stretching onto a surface, thereby forming a coating on that surface.

As such, in some embodiments, the boron nitride-treated polyimide coating material is in the form of a layer on at least a portion of an inner surface of a tubing, a layer on at least a portion of an outer surface of a tubing, or in the form of a layer on at least a portion of an inner and outer surface of a tubing. In some embodiments, the outer surface (outer diameter, OD) of a tubing is at least 50% coated, at least 75% coated, or substantially completely coated, e.g., 100% coated with the disclosed boron nitride-treated polyimide coating material. In some embodiments, the inner surface (inner diameter, ID) of a tubing is at least 50% coated, at least 75% coated, or substantially completely coated, e.g., 100% coated with the disclosed boron nitride-treated polyimide coating material. In some embodiments, each of the inner and outer surfaces is independently at least 50% coated, at least 75% coated, or substantially completely coated, e.g., 100% coated with the disclosed boron nitride-treated polyimide coating material. In some embodiments, one of the inner or outer surfaces of the tubing is uncoated. It is noted that coated tubings as provided herein are not precluded from having other types of coatings associated therewith. For example, a given coated tubing may comprise the boron nitride-treated polyimide coating on one of the inner or outer surfaces and a different type of coating on the other of the inner and outer surfaces. In some embodiments, a different type of coating can be applied on top of the boron nitride-treated polyimide coating on the inner and/or outer surface. In some embodiments, a portion of an inner and/or outer surface comprises the boron nitride-treated polyimide coating and another portion of the inner and/or outer surface can comprise a different type of coating. The different type of coating that can optionally be associated in various ways with a coated tubing as described herein is not particularly limiting and can, in some embodiments, comprise polyimide (not treated with BN).

The liner tubing associated with the coated tubings provided herein is not particularly limited. In some embodiments, the liner tubing is a catheter liner, and the composition of the catheter liner can vary. In some embodiments, the liner tubing can comprise a polymer such as a PTFE, FEP, polyurethane (PU), polyvinyl chloride (PVC) silicone, and combinations thereof. The catheter liners can be of various types and intended for various applications (e.g., including, but not limited to, neurovascular catheters). Advantageously, in some embodiments, the catheter liner comprises a PFAS-free material. Representative sizes of catheter liner tubings generally can vary, with inner diameters (IDs) ranging from 0.001 inches to 0.4 inches (e.g., 0.013″ to 0.4″) and nominal wall thicknesses below 0.004″, e.g., ranging from about 0.0004″ to about 0.004″.

The freestanding tube comprising boron nitride-treated polyimide (or the coated tubing, e.g., catheter liner), can be incorporated within a construction or assembly, such as a catheter assembly. The disclosed boron nitride polyimide-treated tubes/coatings on catheter liners provided herein can provide assemblies/constructions exhibiting excellent lubricity and mechanical strength which are, in some embodiments, comparable to those of fluorinated materials that are currently used in catheter liners and catheter constructions.

For example, in some embodiments, a catheter construction or catheter comprising one or more other layers or components along with the modified catheter liner (comprising the boron nitride-treated polyimide coating described herein) is provided. Non-limiting adjacent component layers (with which the boron nitride-treated polyimide layer can be in direct contact) include, e.g., an adjacent hypotube wall and/or an adjacent polymeric layer, with certain example adjacent components/layers including, but not limited to, outer jackets, braid layers, fiber layers, and/or coil layers. In some embodiments, the catheter construction or catheter comprises a braided or coiled wire reinforcing layer on an outer surface of the modified catheter liner (coated tubing) described herein. In some embodiments, the catheter construction comprises a metallic or polymeric structure overlying and in intimate contact with at least a portion of the modified catheter liner (coated tubing). The metallic or polymeric structure can be, e.g., a metallic or polymeric tube, such as a straight or laser-cut tube or can be a coiled structure. In some embodiments, a catheter construction or catheter is provided that comprises a catheter jacket overlying one or more of these components. In some embodiments, the inner surface (ID) of the modified catheter liner is the catheter construction inner surface and/or the catheter inner surface.

The disclosed coatings can provide one or more surfaces with values suitable for use in catheter assemblies/constructions, including, but not limited to, suitable coefficient of friction (COF) values and/or surface roughness values, while maintaining sufficient mechanical properties, e.g., tensile modulus values. For example, in some embodiments, one or more coated surfaces of the coated tubings or assemblies comprising the coated tubings can exhibit COF, Ra, and/or Rz values as described herein above (and/or in the examples) relative to the freestanding coating tubes.

The method of producing the disclosed coating material, coated products, and constructions/assemblies comprising such coated products can vary. In some embodiments, the boron nitride-treated polyimide coating can be formed by preparing a liquid comprising the desired coating components (e.g., in the form of a dispersion), applying the liquid to a wire, and removing the resulting coating from the underlying wire, where the coating is thus provided in a free-standing tubular form. This tubular form of the coating can then be applied to another substrate, e.g., a tubing (e.g., liner tubing) as described herein to provide the coated tubing.

In some embodiments, the boron nitride-treated polyimide coating is created by first forming a dispersion by adding 5-50 wt. % BN filler to PA. The solution generally further comprise one or more solvents, and the one or more solvents can comprise any solvent in which the components are dispersible. One non-limiting solvent is n-methyl-2-pyrrolidone (NMP). In some embodiments, the PA is directly provided; in other embodiments, various dianhydrides and amines can be used to form the PA, which reaction would be appreciated by one of ordinary skill in the art.

After mixing the filler with the PA, the solution is, in some embodiments, held under vacuum for a given period of time in order to remove trapped air and create a better surface finish (reducing “blisters”). One non-limiting condition for this process is 1.5 hours under vacuum at 25° C. A wire is passed through the BN-PA solution. The wire can be, e.g., a copper or stainless steel wire; in some embodiments, the wire is annealed to soften the metal and produce an oxide layer (e.g., copper oxide) thereon prior to being passed through the solution. During this process of coating the BN-PA solution onto the wire, the solution can be held, e.g., at 25° C.). The coating process generally leaves a very thin coating of the boron nitride-treated polyimide on the surface of the wire (0.0001″-0.0002″ per pass through solution). The coated wire is then subjected to drying and curing, e.g., by passing the coated wire through an oven to dry and cure the coating material. The coated wire may optionally then pass through the solution multiple times to add additional coating thickness. For example, in some embodiments, the wire is passed through the solution only once; in other embodiments, the wire is passed through the solution 2 or more times, 3 or more times, 4 or more times, 5 or more times, 6 or more times, 7 or more times, 8 or more times, 9 or more times, or 10 or more times.

The thickness of this boron nitride-treated polyimide coating on the wire can range from 0.0004″ to 0.005″, e.g., 0.0005″-0.002″. This “coating” thickness thus also describes the average wall thickness of the corresponding freestanding tubes. The wire is then removed, leaving a tube (also referred to as a “freestanding coating” or just a “tube”) comprising the boron nitride-treated polyimide material. This freestanding coating or tube can have an inner diameter, e.g., of about 0.0043 inches to 0.091 inches, with a nominal wall thickness ranging from 0.0004 inches to 0.005 inches.

In some embodiments, the boron nitride-treated polyimide freestanding coating is applied to just the ID or just the OD of the tubing (e.g., liner) to produce the coated tubing, e.g., to add lubricity where it is needed. This strategy can, in some embodiments, add overall mechanical strength to the tubing while also giving a bondable surface (polyimide) on either the ID or OD of the tubing (which, as noted above, can be further associated with one or more additional components, e.g., in a catheter assembly/construction). In some embodiments, independent boron nitride-treated polyimide freestanding coatings are applied to both the inner and outer diameters of the tubing (e.g., liner). Such coatings can be substantially the same or can be different.

In some embodiments, the coated wire described herein above is rinsed and residual solvents are removed before or after the coated wire is collected onto a spool, then spooled-off, stretched, then cut, a process in which the elastic boron nitride-treated polyimide coating snaps backs to its original shape and is removed from the wire, leaving the freestanding boron nitride-treated polyimide freestanding coating in the form of a tube with a certain inner diameter and wall thickness, which can readily be applied to inner and/or outer surfaces of tubings as described herein to provide the disclosed boron nitride-treated polyimide coated tubings.

### Experimentals

Aspects of the present disclosure are more fully illustrated by the following examples, which are set forth to illustrate certain aspects of the present invention and are not to be construed as limiting thereof.

Different particle sizes and particle shapes of BN were obtained from a variety of suppliers as shown in Table 1. In particular, two particle shape variants of the hexagonal Boron Nitride were obtained: the first shape having a smaller particle size known as platelets (Examples 1, 2, 3, and 4) and the second shape having a larger particle size referred to as agglomerate (Example 5 & 6). Using NX1 Momentive Technologies boron nitride with the smallest particle size (0.9 d50 μm) and platelet particle shape (Example 1), BN-PI tubes were prepared at the following concentrations: 5% (Example 1a), 10% (Example 1b), 15% (Example 1c), 20% (Example 1d), and 25% (Example 1e) according to the BN-PI compound preparation outlined herein, and the COF, surface roughness, and tensile mechanical properties were evaluated for each example according to the methods outlined herein and compared to the COF, surface roughness, and tensile mechanical properties of a PI tube (I.S.T.) most commonly used in legacy processing with no filler (Comparative Example 1) and a PI Glide™ tube (Zeus) (Comparative Example 2) with a PTFE filler for enhanced lubricity. Additional material information can be found in Tabl1 1 for these comparative examples. All samples were produced targeting the same tube size ID: 0.032″, OD: 0.034″, and a wall thickness of 0.001″ for optimal comparison as shown in Table 2.

It was determined that the Example 1c 15% NX1 Momentive Technologies boron nitride achieved an optimal combination of lubricity, surface roughness, and mechanical properties; therefore, it was determined that all the different particle sizes and particle shapes of BN obtained from a variety of suppliers would be evaluated for their COF, surface roughness, and tensile mechanical properties at a 15% concentration and compared to the respective properties of the PI tube (Comparative Example 1) and a PI Glide™ tube (Comparative Example 2). Results of this comparison are shown in Table 3. All trials were conducted under identical environmental and processing conditions to ensure consistency and comparability across samples.

Images of the tubing specimens were obtained a via Keyence VHX-7100 (serial number V5342030262) digital microscope at 40× magnification.

The polyamic acid-boron nitride (BN-PI) for each example was prepared by first determining the weight of boron nitride powder needed to produce a final polyimide tubular coating with a certain percent of boron nitride by weight. The powder was added to the commercially available polyamic acid in n-methyl-2-pyrrolidone (NMP), then stirred via spatula until the powder dispersed uniformly. Once no loose powder was observed in the polyamic acid, the mixture was then placed under vacuum for at least 45 minutes at −1 Bar to remove residual air pockets in the resin produced from stirring. Tubes were formed as described herein above. The COF, surface roughness, and tensile mechanical properties were evaluated for each example freestanding tube.

The COF was determined using a Harland COF FTS7000 Friction Testing System equipped with 60A silicone pads, 1000 g load cell, Temperature 37° C. using wet media and a clamp force of 200 g. Ten separate tubes per sample were tested, number of cycles=3 per tube, (total 30 readings per sample) were tested at a velocity of 10 mm/s over a length of 60 mm. The average of all ten samples is reported.

The surface roughness was evaluated using a Keyence VK-X 3000 confocal laser microscope, serial number 6DOM000031. Images were taken at 50× magnification and processed using the Keyence Multi File Analyzer application. Image processing was used to digitally flatten the image to remove the curvature from the tube shape. The “surface shape correction-cylinder” tool was used to do this. The roughness was measured using the built “in line roughness” tool which simulates what a physical profilometer would measure if one were used. The virtual stylus was set to a 2 micrometer diameter and a 60 degree tip angle. Each reading was an average of one reading each from 3 tubes from that batch, measured in the machine direction, along the long axis of the tube, and the unit of measurement is microinches (pin).

The tensile mechanical properties were evaluated using an Instron 5965 machine equipped with grips configured to securely hold the tubular material and 1 kN Load cell according to the following test method: DEV Method 58_ASTM D638 Tensile Properties of Plastics Tubing. Each specimen was prepared to provide sufficient length for engagement in the grips while maintaining a test gauge length distance of 2 inches. The samples were then tested at a rate of 2 inches per minute. Each sample was tested 5 times, and average modulus (Ksi), percent elongation, and tensile strength (psi) was reported.

TABLE 1
Sample Identification

| | | | | | Particle Size |
|---|---|---|---|---|---|
| Example | Material Type | Supplier | Grade | Shape | (d50 μm)¹ |
| Comparative 1 | Polyimide | IST | Rc5019 | NA | NA |
| Comparative 2 | PTFE Filled Polyimide | IST | Rc5019T | NA | NA |
| Example 1 | Boron Nitride | Momentive Technologies | NX1 | Platelets | 0.9 |
| Example 2 | Boron Nitride | Saint-Gobain | SP2 | Platelets | 2.2 |
| Example 3 | Boron Nitride | Saint-Gobain | SP5 | Platelets | 5.5 |
| Example 4 | Boron Nitride | Saint-Gobain | SP30 | Platelets | 30 |
| Example 5 | Boron Nitride | Saint-Gobain | SA75 | Agglomerate | 75 |
| Example 6 | Boron Nitride | Momentive Technologies | AC6028 | Agglomerate | 100-150* |

¹= Published value from supplier.
*Taken from third party published literature.

TABLE 2
Loading Trials with Properties

| | | Coefficient | Surface Roughness Machine | | Tensile Properties | | |
|---|---|---|---|---|---|---|---|
| | | of friction | Direction, Single Line | | | | Tensile |
| | Loading | (COF) | Ra | Rz | Modulus | Elongation | Stength |
| Entry | % | COF Avg | (μin) | (μin) | (Ksi) | (%) | (psi) |
| Comparative 1 | NA | 2.07 ± 0.07 | 1.01 ± 1.1 | 40.9 ± 65.2 | 462 ± 46 | 56 ± 4 | 27,250 ± 2,656 |
| Comparative 2 | NA | 1.25 ± 0.07 | 22.5 ± 6.0 | 134.7 ± 24.2 | 243 ± 12 | 34 ± 4 | 11,410 ± 387 |
| Example 1a | 5 | 1.55 ± 0.02 | 3.7 ± 1.7 | 25.5 ± 15.8 | 414 ± 13 | 64 ± 9 | 22,440 ± 1,584 |
| Example 1b | 10 | 1.26 ± 0.21 | 4.8 ± 0.9 | 35.8 ± 7.9 | 426 ± 9 | 60 ± 8 | 19,290 ± 1,082 |
| Example 1c | 15 | 1.17 ± 0.18 | 7.3 ± 0.3 | 67.9 ± 20.5 | 456 ± 33 | 52 ± 2 | 16,800 ± 1,056 |
| Example 1d | 20 | 1.07 ± 0.10 | 6.6 ± 2.2 | 49 ± 14.2 | 497 ± 31 | 45 ± 4 | 15,440 ± 544 |
| Example 1e | 25 | 0.98 ± 0.10 | 11 ± 4.0 | 66.2 ± 22.1 | 525 ± 32 | 33 ± 5 | 13,530 ± 351 |

TABLE 3
15% BN-PI Property Results

| | Coefficient | Surface Roughness Machine | | Tensile Properties | | |
|---|---|---|---|---|---|---|
| | of friction | Direction, Single Line | | | | Tensile |
| | (COF) | Ra | Rz | Modulus | Elongation | Stength |
| Entry Number | COF Avg | (μin) | (μin) | (Ksi) | (%) | (psi) |
| Comparative 1 | 2.07 ± 0.07 | 1.01 ± 1.1 | 40.9 ± 65.2 | 462 ± 46 | 56 ± 4 | 27,250 ± 2,656 |
| Comparative 2 | 1.25 ± 0.07 | 22.5 ± 6.0 | 134.7 ± 24.2 | 243 ± 12 | 34 ± 4 | 11,410 ± 387 |
| Example 1c | 1.17 ± 0.18 | 7.3 ± 0.3 | 67.9 ± 20.5 | 456 ± 33 | 52 ± 2 | 16,800 ± 1,056 |
| Example 2 | 1.23 ± 0.08 | 7.5 ± 0.9 | 61.9 ± 15.3 | 478 ± 21 | 37 ± 3 | 15,550 ± 642 |
| Example 3 | 1.25 ± 0.08 | 12.1 ± 4.5 | 83.5 ± 24.4 | 514 ± 40 | 38 ± 3 | 15,480 ± 1,063 |
| Example 4 | 1.29 ± 0.10 | 19.6 ± 8.0 | 126.8 ± 59.3 | 472 ± 66 | 19 ± 5 | 16,290 ± 1,610 |
| Example 5 | 1.38 ± 0.06 | 56 ± 48 | 307.8 ± 325.4 | 413 ± 35 | 22 ± 3 | 16,940 ± 1,364 |
| Example 6 | 1.20 ± 0.12 | 0.8 ± .02 | 5.2 ± 1.6 | 452 ± 40 | 33 ± 8 | 22,210 ± 968 |

The results of the loading trial as shown in Table 2 revealed a distinct relationship between the loading percentage of boron nitride and the COF: as the percent loading of BN increased, the surface roughness increased (while at its highest still being roughly half the surface roughness of PI Glide™), and the COF values decreased. Example 1a of the 5% BN-PI tubing exhibited a COF of approximately 1.55±0.02 and for each subsequent example (Example 1b-1e), the COF decreased resulting in the COF of 25% BN-PI being 0.98±0.10, indicating that lubricity and BN loading have a direct relationship within the BN concentration range tested. A similar relationship was observed for tensile modulus; however, tensile strength showed that strength decreases as the BN loading increases but the tensile strength for all BN-PI samples was still significantly higher than the tensile strength of PI Glide™. with the lowest loading of BN resulting in almost twice the tensile strength of PI Glide™ and roughly twice the modulus suggesting that the addition of BN does not compromise the mechanical properties of PI as much as the addition of PTFE filler does. This is a particularly surprising result. Every loading of BN-PI resulted in a COF significantly lower (COFs from 0.98±0.10 to 1.55±0.02) than that of comparative example 1 (PI) (COF of 2.07±0.07). Examples 1b, 1c, 1d, & 1e achieved COFs either equal to or significantly lower than the COF of PI Glide™. This suggests that using a concentration greater than 10% BN achieves comparable or significantly better lubricity than that achieved by current PI-PTFE market standards (PI Glide™).

The surface roughness of all BN-PI samples was significantly higher than the surface roughness of PI on its own (1.01±1.1 μin) and increased as BN loading increased. One common approach in the industry for enhancing lubricity is to increase surface roughness. Therefore, the surface roughness comparison between the BN-PI samples and the PI Glide™ sample is of particular note. The surface roughnesses of all BN-PI samples (Ra=3.7±1.7 to 11±4.0 pin, Rz=25.5±15.8 to 66.2±22.1 pin) were significantly lower than that of the PI Glide™ sample (Ra=22.5±6.0 pin), and it is particularly surprising that in light of their significantly lower surface roughnesses that every BN-PI sample was able to achieve comparable or significantly lower COFs than PI Glide™. The reduced COF observed for PI Glide™ can be attributed to the inherently lubricious nature of PTFE and its characteristically low surface energy; however, no such relationship to lubricity and BN has been demonstrated, and it is particularly surprising that BN is able to achieve significantly lower COFs while maintaining a significantly lower surface roughness than the industry standard of a PTFE filler.

Furthermore, the tensile modulus of PI Glide™ (243±12 Ksi) was shown to be roughly half that of PI (462±46 Ksi), while the tensile modulus of all BN-PI samples (414±13-525±32 Ksi) was comparable to or higher than the tensile modulus of PI demonstrating that using BN instead of a typical fluorinated lubricity additive such as PTFE filler with PI surprisingly achieves not only comparable or significantly better lubricity than PI Glide™, but also significantly higher modulus than PI Glide™ and a comparable or better modulus than PI on its own. These results demonstrate that BN is a PFAS-free solution for enhancing the lubricity of PI without compromising the mechanical properties of PI and, in fact, BN provides a path to achieving maximized lubricity and maximized modulus, a balance not achieved by PI or PI Glide™. The present disclosure provides tubing that enables the retention of a modulus comparable to that of unfilled polyimide while simultaneously delivering lubricity characteristics like those of PI Glide™ This disclosure overcomes the longstanding trade-off between lubricity and mechanical properties that exists for fluorinated solutions by employing boron nitride as a filler that maintains both without sacrificing performance.

Regarding the tensile properties, it should be noted that modulus can be optimized for catheter flexibility, so in finding the balance between optimal lubricity, modulus, strength, and elongation, it is important to consider what modulus range corresponds best to the flexibility needs of a particular catheter delivery system and what BN loading level enables optimized processing. Therefore, a 15% concentration of BN was deemed as a preferred loading for the BN-PI samples where a variety of boron nitride particles from different suppliers with varying particle sizes and shapes were tested.

As shown in Table 3, for tubing produced using platelets, no significant variation in the COF was observed as a function of particle size, and the COF results for these samples were significantly lower than PI on its own and were comparable or lower than the COF of the industry benchmark PI Glide™. It was observed that particle size did impact surface roughness as, in general, surface roughness increased as particle size increased independent particle shape. In example 6, the agglomerate particles of the largest particle size tested dispersed particularly poorly during processing thus impacting the uniformity of the coating and the surface roughness results. It was deemed that platelet-type BN fillers with particle sizes of 0.9-30 μm (Examples 1c, 2, 3, and 4) yielded the most desirable COF results comparable to that of PI Glide™ and the surface roughness results for examples 1c and 2 (the lowest particle sizes tested) were the lowest. No significant differences in tensile modulus, elongation, and tensile strength were observed in general for all BN-PI samples from different suppliers except for example 6. Overall, platelet fillers with the smallest particle sizes provided the most favorable combination of low friction, controlled surface morphology, and mechanical robustness. These results indicate that when all tested properties are taken into account BN enhances lubricity independent of supplier, particle size and shape, and that platelet type BN with lower particle sizes may be preferred for finding the optimal balance of lubricity, surface roughness, flexibility, and strength. Microscopy of the samples confirmed this as well. All BN-PI samples in Table 3 achieved better lubricity and comparable mechanical properties to PI on its own while achieving a much higher modulus than PI Glide™.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A tube comprising boron nitride-treated polyimide, the tube having an inner surface and an outer surface,
wherein tube exhibits:
a) a coefficient of friction (COF) less than 1.6 on the inner surface and the outer surface;
b) a Ra surface roughness less than 12 microinches on the inner surface and the outer surface;
c) a Rz surface roughness less than 70 microinches on the inner surface and the outer surface; and
d) a tensile modulus between 400-600 Ksi.

2. The tube of claim 1, wherein the tube is substantially homogeneous in composition.

3. The tube of claim 1, comprising about 5% to about 25% boron nitride by weight, based on a weight of the tube.

4. The tube of claim 1, comprising about 10% to about 20% boron nitride by weight, based on a weight of the tube.

5. The tube of claim 1, having an average wall thickness of 0.0005 to 0.002″.

6. The tube of claim 1, consisting essentially of the boron nitride-treated polyimide.

7. The tube of claim 1, further comprising one or more fillers.

8. The tube of claim 1, wherein the COF is less than 1.3.

9. The tube of claim 1, wherein the COF is less than 1.2.

10. A catheter comprising the tube of claim 1 on an inner diameter thereof.

11. A coated liner, comprising:
a liner tubing; and
a coating comprising the tube of claim 1 on at least a portion of an inner surface of the liner tubing and/or the outer surface of the liner tubing.

12. The coated liner of claim 11, wherein the tube is in a radially unstretched form.

13. The coated liner of claim 11, wherein the tube is in a radially stretched form.

14. The coated liner of claim 11, wherein the liner tubing comprises a non-fluorinated polymeric material.

15. The coated liner of claim 11, wherein the coating is present only on at least a portion of the inner surface of the liner tubing.

16. The coated liner of claim 11, wherein the coating is present only on at least a portion of the outer surface of the liner tubing.

17. The coated liner of claim 11, wherein the coating is present on at least a portion of the outer surface of the liner tubing and at least a portion of the inner surface of the liner tubing.

18. The coated liner of claim 11, wherein the coating is present on substantially all of the inner surface of the liner tubing and/or the outer surface of the liner tubing.

19. The coated liner of claim 11, further comprising one or more different coatings in addition to the coating comprising boron nitride-treated polyimide.

20. A catheter comprising the coated liner of claim 11 on an inner diameter thereof.
